(19) United States
(12) Patent Application Publication
(10) Pub. No.: US 2016/0375234 A1
(43) Pub. Date: Dec. 29, 2016

(54) TRANSPAPILLARY METHODS AND COMPOSITIONS FOR DIAGNOSING AND TREATING BREAST CONDITIONS

(71) Applicant: ATOSSA GENETIC INC., Seattle, WA (US)

(72) Inventor: Steven C. Quay, Seattle, WA (US)

(21) Appl. No.: 15/110,718

(22) PCT Filed: Jan. 9, 2015
(86) PCT No.: PCT/US15/10808

§ 371 Date: Jul. 8, 2016

Related U.S. Application Data
(60) Provisional application No. 61/926,180, filed on Jan. 10, 2014.

(51) Int. Cl. A61M 37/00; A61B 6/00; A61B 5/055; A61B 8/08; A61F 13/14; A61K 51/00; A61K 31/19; A61K 31/704; A61K 31/337; A61K 31/513; A61K 49/00; A61M 31/00; A61K 31/138

(52) U.S. Cl. CPC A61M 37/00; A61M 31/005; A61B 6/481; A61B 5/055; A61B 8/481; A61F 13/14; A61K 31/138; A61K 31/19; A61K 31/704; A61K 31/337; A61K 31/513; A61K 49/0002; A61K 51/00

(57) ABSTRACT

Methods and treatments are taught for the diagnosis and treatment of breast conditions, including proliferative breast disease, ductal hyperplasia, lobular hyperplasia, atypical ductal hyperplasia, atypical lobular hyperplasia, ductal carcinoma in situ, lobular carcinoma in situ, lobular carcinoma and invasive breast cancer. The methods and compositions deliver efficacious formulations of chemical and/or biological treatment medicaments to the breast via a transpapillary route.

### CROSS REFERENCE

This application claims the benefit of U.S. Provisional Application No. 61/926,180 filed Jan. 10, 2014, which is incorporated by reference herein in its entirety.

### BACKGROUND

Breast disorders include breast cancers and benign lesions, such as ductal hyperplasia, lobular hyperplasia, atypical ductal hyperplasia, and atypical lobular hyperplasia. Breast cancers include any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer.

Current best practice for the treatment of breast cancer is to diagnose breast cancer with mammography and then treat the patient with surgery, radiation therapy, and chemotherapy. There exists a need for improved methods for treating breast conditions such as breast cancer.

### SUMMARY OF THE INVENTION

Disclosed herein, in certain embodiments, are methods of delivering a composition to a breast duct of an individual in need thereof, comprising: (a) contacting a composition contained within a treatment chamber of a device with a nipple of a breast; and (b) applying positive pressure on the composition. In some embodiments, the composition is forced into the breast duct due to the positive pressure. In some embodiments, the device further comprises: a first opening sized to circumscribe a nipple, which opening is operatively connected to the treatment chamber. In some embodiments, the device further comprises: a second opening operatively connected to the treatment chamber through which through which the composition is instilled into the treatment chamber. In some embodiments, the device further comprises a third opening operatively connected to the treatment chamber through which positive pressure is applied to the composition. In some embodiments, the composition comprises at least one therapeutic agent. In some embodiments, the composition comprises a plurality of therapeutic agents. In some embodiments, the composition comprises at least one therapeutic agent selected from the group consisting of: an anthracycline, a platinum agent, a taxane, or combinations thereof. In some embodiments, the composition comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, toremifene, trastuzumab, vinorelbine, and combinations thereof. In some embodiments, the composition comprises 4-hydroxytamoxifen. In some embodiments, the composition comprises tamoxifen. In some embodiments, the composition comprises N-desmethyltamoxifen. In some embodiments, the composition comprises cis-tamoxifen. In some embodiments, the composition comprises butyric acid. In some embodiments, the composition comprises doxorubicin. In some embodiments, the composition comprises epirubicin. In some embodiments, the composition comprises paclitaxel. In some embodiments, the composition comprises docetaxel. In some embodiments, the composition comprises fluorouracil. In some embodiments, the composition comprises at least one diagnostic agent. In some embodiments, the composition comprises a plurality of diagnostic agents. In some embodiments, the composition comprises a diagnostic agent selected from a fluorescent agent, a contrast agent and a radionuclide. In some embodiments, the composition comprises a fluorescent agent selected from the group consisting of: a fluorescein dye, a rhodamine dye and a cyanine dye. In some embodiments, composition comprises a diagnostic agent selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE680, Alexa Fluor 750, IRDye800CW, ICG. In some embodiments, the composition comprises a contrast agent selected from a radiocontrast agent, MRI contrast agent, or ultrasound contrast agent. In some embodiments, the composition comprises a contrast agent selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof. In some embodiments, the composition comprises a radionuclide selected from the group consisting of: 211At, 131I, 125I, 90Y, 186Re, 188Re, 153Sm, 212Bi, 32P, 64Cu, a radioactive isotope of Lu, or any combinations thereof. In some embodiments, the methods further comprise detecting the diagnostic agent. In some embodiments, the composition has a low viscosity. In some embodiments, the composition has a viscosity of less than 10 cp, 5 cp, or 1 cp at 25° C. In some embodiments, the composition comprises dissolved carbon dioxide. In some embodiments, the composition is stored between 0° C. and 20° C. In some embodiments, the positive pressure is applied to the composition by the escape of the carbon dioxide from the composition as the temperature of the composition increases. In some embodiments, the composition is contacted with the nipple of a breast on the 2nd week of the individual's menstrual cycle. In some embodiments, the composition is contacted with the nipple of a breast for at least 6 hrs, 8 hrs, 10 hrs, 12 hrs, 18 hours, or 24 hours. In some embodiments, the methods further comprise adhering the device to the nipple. In some embodiments, the device further comprises an adhesive which adheres the device to the breast. In some embodiments, the methods further comprise applying a topical anesthetic to the nipple before the composition is contacted with the nipple. In some embodiments, the methods further comprise cleaning the nipple before the composition is contacted with the nipple. In some embodiments, the methods further comprise applying a cover over the nipple after removing the device. In some embodiments, the cover is waterproof and/or airtight. In some embodiments, the cover comprises a liquid bandage. In some embodiments, the cover comprises a patch. In some embodiments, the cover comprises a film. In some embodiments, the cover comprises an occlusive agent. In some embodiments, the cover comprises an anti-inflammatory agent or an antiseptic.

Disclosed herein, in certain embodiments, are methods of treating a breast disorder, comprising: (a) contacting a treatment chamber comprising a composition comprising at least one therapeutic agent with a nipple of a breast; and (b) applying positive pressure on the composition comprising at least one therapeutic agent. In some embodiments, the breast disorder is a breast cancer. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma. In some embodiments, the composition comprising at least one therapeutic agent is forced into the breast duct due to the positive pressure. In some embodiments, the device further comprises: (a) a first opening sized to circumscribe a nipple, which opening is operatively connected to the treatment chamber; and (b) a second opening operatively connected to the treatment chamber through which positive pressure is applied to the composition comprising at least one therapeutic agent. In some embodiments, the device further comprises a third opening through which the composition comprising at least one therapeutic agent is instilled into the treatment chamber. In some embodiments, the composition comprises a plurality of therapeutic agents. In some embodiments, the at least one therapeutic agent is selected from the group consisting of: an anthracycline, a platinum agent, a taxane, or combinations thereof. In some embodiments, the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, and combinations thereof. In some embodiments, the at least one therapeutic agent is hydroxytamoxifen. In some embodiments, the composition comprises tamoxifen. In some embodiments, the composition comprises N-desmethyltamoxifen. In some embodiments, the composition comprises cis-tamoxifen. In some embodiments, the at least one therapeutic agent is butyric acid. In some embodiments, the at least one therapeutic agent is doxorubicin. In some embodiments, the at least one therapeutic agent is epirubicin. In some embodiments, the at least one therapeutic agent is paclitaxel. In some embodiments, the at least one therapeutic agent is docetaxel. In some embodiments, the at least one therapeutic agent is fluorouracil. In some embodiments, the methods further comprise sealing the device to the nipple. In some embodiments, the methods further comprise cleaning the nipple before the treatment chamber is contacted with the nipple. In some embodiments, the methods further comprise applying a cover over the nipple after removing the device.

Disclosed herein, in certain embodiments, are methods of diagnosing a disorder of a breast in an individual in need thereof, comprising: (a) contacting a treatment chamber comprising a composition comprising a diagnostic agent with a nipple of a breast; and (b) applying positive pressure on the composition comprising a diagnostic agent. In some embodiments, the composition comprising a diagnostic agent is forced into the breast duct due to the positive pressure. In some embodiments, the device further comprises: (a) a first opening sized to circumscribe a nipple, which opening is operatively connected to the treatment chamber; and (b) a second opening operatively connected to the treatment chamber through which positive pressure is applied to the composition comprising a diagnostic agent. In some embodiments, the device further comprises a third opening through which the composition comprising a diagnostic agent is instilled into the treatment chamber. In some embodiments, the diagnostic agent is selected from a fluorescent agent, a contrast agent and a radionuclide. In some embodiments, the fluorescent agent is selected from the group consisting of: a fluorescein dye, a rhodamine dye and a cyanine dye. In some embodiments, diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE710, Alexa Fluor 750, IRDye800CW, ICG. In some embodiments, the contrast agent is a radiocontrast agent, MRI contrast agent, or ultrasound contrast agent. In some embodiments, the contrast agent is selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof. In some embodiments, the radionuclide is selected from the group consisting of: 211At, 131I, 125I, 90Y, 186Re, 188Re, 153Sm, 212Bi, 32P, 64Cu, a radioactive isotope of Lu, or any combinations thereof. In some embodiments, the methods further comprise detecting the diagnostic agent. In some embodiments, the methods further comprise sealing the device to the nipple. In some embodiments, the methods further comprise cleaning the nipple before the treatment chamber is contacted with the nipple. In some embodiments, the methods further comprise applying a cover over the nipple after removing the device.

Disclosed herein, in certain embodiments, are compositions for use in the treatment or diagnosis of a breast cancer, comprising (a) at least one therapeutic agent or a diagnostic agent, and (b) a dissolved gas. In some embodiments, the dissolved gas is carbon dioxide. In some embodiments, the composition has a low viscosity. In some embodiments, the composition has a viscosity of less than 10 cp, 5 cp, or 1 cp at 25° C. In some embodiments, the compositions further comprise a plurality of therapeutic agents. In some embodiments, the at least one therapeutic agent is selected from the group consisting of: an anthracycline, a platinum agent, a taxane, or combinations thereof. In some embodiments, the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, and combinations thereof. In some embodiments, the at least one therapeutic agent is 4-hydroxytamoxifen. In some embodiments, the at least one therapeutic agent is tamoxifen. In some embodiments, the at least one therapeutic agent is N-desmethyltamoxifen. In some embodiments, the at least one therapeutic agent is cis-tamoxifen. In some embodiments, the at least one therapeutic agent is butyric acid. In some embodiments, the at least one therapeutic agent is doxorubicin. In some embodiments, the at least one therapeutic agent is epirubicin. In some embodiments, the at least one therapeutic agent is paclitaxel. In some embodiments, the at least one therapeutic agent is docetaxel. In some embodiments, the at least one therapeutic agent is fluorouracil. In some embodiments, the diagnostic agent is selected from a fluorescent agent, a contrast agent and a radionuclide. In some embodiments, the fluorescent agent is selected from the group consisting of: a fluorescein dye, a rhodamine dye and a cyanine dye. In some embodiments, diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE850, Alexa Fluor 750, IRDye800CW, ICG. In some embodiments, the contrast agent is a radiocontrast agent, MRI contrast agent, or ultrasound contrast agent. In some embodiments, the contrast agent is selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof. In some embodiments, the radionuclide is selected from the group consisting of: 211At, 131I, 125I, 90Y, 186Re, 188Re, 153Sm, 212Bi, 32P, 64Cu, a radioactive isotope of Lu, or any combinations thereof. In some embodiments, the composition is stored between 0° C. and 20° C.

Disclosed herein, in certain embodiments, are devices for delivering a composition to a breast duct of an individual in need thereof, comprising: (a) a treatment chamber; (b) a first opening sized to circumscribe a nipple, which opening is operatively connected to the treatment chamber; and (c) a composition comprising at least one therapeutic agent or a diagnostic agent. In some embodiments, the composition comprising the at least one therapeutic agent or the diagnostic agent is contained within the treatment chamber. In some embodiments, the devices further comprise a second opening operatively connected to the treatment chamber through which through which the composition is instilled into the treatment chamber. In some embodiments, the devices further comprise a third opening operatively connected to the treatment chamber through which positive pressure is applied to the composition. In some embodiments, the composition comprises a plurality of therapeutic agents. In some embodiments, the at least one therapeutic agent is selected from the group consisting of: an anthracycline, a platinum agent, a taxane, or combinations thereof. In some embodiments, the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, toremifene, trastuzumab, vinorelbine, and combinations thereof. In some embodiments, the at least one therapeutic agent is hydroxytamoxifen. In some embodiments, the at least one therapeutic agent is tamoxifen. In some embodiments, the at least one therapeutic agent is N-desmethyltamoxifen. In some embodiments, the at least one therapeutic agent is cis-tamoxifen. In some embodiments, the at least one therapeutic agent is butyric acid. In some embodiments, the at least one therapeutic agent is doxorubicin. In some embodiments, the at least one therapeutic agent is epirubicin. In some embodiments, the at least one therapeutic agent is paclitaxel. In some embodiments, the at least one therapeutic agent is docetaxel. In some embodiments, the at least one therapeutic agent is fluorouracil. In some embodiments, the composition comprises a plurality of diagnostic agents. In some embodiments, the diagnostic agent is selected from a fluorescent agent, a contrast agent and a radionuclide. In some embodiments, the fluorescent agent is selected from the group consisting of: a fluorescein dye, a rhodamine dye and a cyanine dye. In some embodiments, diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE680, Alexa Fluor 750, IRDye800CW, ICG. In some embodiments, the contrast agent is a radiocontrast agent, MRI contrast agent, or ultrasound contrast agent. In some embodiments, the contrast agent is selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof. In some embodiments, the radionuclide is selected from the group consisting of: 211At, 1311, 1251, 90Y, 186Re, 188Re, 153Sm, 212Bi, 32P, 64Cu, a radioactive isotope of Lu, or any combinations thereof. In some embodiments, the composition has a low viscosity. In some embodiments, the composition has a viscosity of less than 10 cp, 5 cp, or 1 cp at 25° C. In some embodiments, the composition comprises dissolved carbon dioxide. In some embodiments, the devices further comprise an adhesive which adheres the device to the breast.

### DETAILED DESCRIPTION OF THE INVENTION

Current best practice for the treatment of breast cancer is to diagnose breast cancer with mammography and then to cut, burn, and poison the patient (surgery, radiation therapy, and chemotherapy). That is, local surgery, local radiation therapy, but systemic chemotherapy.

Systemic chemotherapy is accompanied by often severe side-effects. These side effects include, but are not limited to, hair loss, mouth sores, nausea and vomiting, neutropenia, premature menopause, infertility, neuropathy, cardiomyopathy, Hand-foot syndrome, myelodysplastic syndrome, and acute myeloid leukemia.

Proliferative breast disease (PBD), including ductal hyperplasia, lobular hyperplasia, atypical ductal hyperplasia, atypical lobular hyperplasia, ductal carcinoma in situ, and lobular carcinoma, is difficult to diagnosis by current imaging methods because it involves such small numbers of cells that even the most modern imaging methods fail to detect it.

With respect to treatment, local, effective, easy-to-administer diagnostic and chemotherapy would make early diagnosis possible and obviate the side effects of systemic treatment and could produce higher levels of drugs in the breast, improving efficacy.

Intraductal treatment with pharmaceuticals has been shown to be both effective and with very little drug reaching the blood stream, reducing side effects. The challenge is being able to cannulate the correct duct and there is sometimes considerable pain.

Active, transpapillary methods have been developed using iontophoresis. These methods involve application of an electric current to the breast which ‘conducts’ a drug into the ducts of the breast. This method often results in discomfort to the patient and is limited to drugs which have a net charge.

Passive, transpapillary methods have been tried but to date there have been no studies to demonstrate these would be efficacious in humans

There exists a need for a locally acting medicament for the diagnosis and treatment of breast conditions.

Disclosed herein, in certain embodiments, are methods of delivering a composition to a breast duct of an individual in need thereof, comprising: (a) contacting a composition contained within a treatment chamber of a device with a nipple of a breast; and (b) applying positive pressure on the composition. In some embodiments, the composition is forced into the breast duct due to the positive pressure. In some embodiments, the device further comprises: a first opening sized to circumscribe a nipple, which opening is operatively connected to the treatment chamber. In some embodiments, the device further comprises: a second opening operatively connected to the treatment chamber through which through which the composition is instilled into the treatment chamber. In some embodiments, the device further comprises a third opening operatively connected to the treatment chamber through which positive pressure is applied to the composition. In some embodiments, the composition comprises at least one therapeutic agent. In some embodiments, the composition comprises a plurality of therapeutic agents. In some embodiments, the composition comprises at least one diagnostic agent. In some embodiments, the methods further comprise detecting the diagnostic agent. In some embodiments, the composition has a low viscosity. In some embodiments, the composition has a viscosity of less than 10 cp, 5 cp, or 1 cp at 25° C. In some embodiments, the composition comprises dissolved carbon dioxide. In some embodiments, the composition is stored between 0° C. and 20° C. In some embodiments, the positive pressure is applied to the composition by the escape of the carbon dioxide from the composition as the temperature of the composition increases. In some embodiments, the composition is contacted with the nipple of a breast on the 2^{nd} week of the individual's menstrual cycle. In some embodiments, the composition is contacted with the nipple of a breast for at least 6 hrs, 8 hrs, 10 hrs, 12 hrs, 18 hours, or 24 hours. In some embodiments, the methods further comprise adhering the device to the nipple. In some embodiments, the device further comprises an adhesive which adheres the device to the breast. In some embodiments, the methods further comprise cleaning the nipple before the medicament is contacted with the nipple. In some embodiments, the methods further comprise applying a cover over the nipple after removing the device. In some embodiments, the cover is waterproof and/or airtight. In some embodiments, the cover is a liquid bandage. In some embodiments, the cover is a patch. In some embodiments, the cover comprises an anti-inflammatory agent or an antiseptic.

Disclosed herein, in certain embodiments, are methods of treating a breast cancer, comprising: (a) contacting a composition comprising at least one therapeutic agent contained within a treatment chamber of a device with a nipple of a breast; and (b) applying positive pressure on the composition comprising at least one therapeutic agent. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

Disclosed herein, in certain embodiments, are methods of diagnosing a disorder of a breast in an individual in need thereof, comprising: (a) contacting a composition comprising a diagnostic agent contained within a treatment chamber of a device with a nipple of a breast; and (b) applying positive pressure on the composition comprising a diagnostic agent. In some embodiments, the breast cancer is a benign breast lesion. In some embodiments, the benign breast lesion is ductal hyperplasia, lobular hyperplasia, atypical ductal hyperplasia, or atypical lobular hyperplasia. In some embodiments, the breast disorder is a breast cancer. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

### Breast Disorders

As used herein, “breast disorder” means any disorder of a breast. Breast disorders include benign lesions of the breast and breast cancer. Benign breast lesions include, but are not limited to, ductal hyperplasia, lobular hyperplasia, atypical ductal hyperplasia, and atypical lobular hyperplasia.

As used herein, “breast cancer” means any malignant tumor of breast cells. There are several types of breast cancer. Exemplary breast cancers include, but are not limited to, ductal carcinoma in situ, lobular carcinoma in situ, invasive (or infiltrating) ductal carcinoma, invasive (or infiltrating) lobular carcinoma, inflammatory breast cancer, triple-negative breast cancer, ER+ breast cancer, HER2+ breast cancer, adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, and micropapillary carcinoma. A single breast tumor can be a combination of these types or be a mixture of invasive and in situ cancer.

Ductal hyperplasia is hyperplasia of a breast duct, not accompanied by histomorphologic abnormalities. Ductal hyperplasia is not usually considered predicative of a predisposition for breast cancer.

Lobular hyperplasia is hyperplasia of a breast lobule, not accompanied by histomorphologic abnormalities. Lobular hyperplasia is not usually considered predicative of a predisposition for breast cancer.

Atypical ductal hyperplasia (ADH) is a benign lesion of the breast characterized by hyperplasia of at least one breast duct and histomorphologic abnormalities. While not cancerous, ADH may be indicative of a predisposition for breast cancer. ADH may be excised by lumpectomy.

Atypical lobular hyperplasia is a benign lesion of the breast characterized by hyperplasia of a breast lobule and histomorphologic abnormalities. While not cancerous, ADH may be indicative of a predisposition for breast cancer. ADH may be excised by lumpectomy.

Ductal carcinoma in situ (DCIS) is the most common non-invasive breast cancer. It involves the cells lining the breast ducts. In DCIS, the cells have not spread beyond the walls of the ducts into the surrounding breast tissue. About 1 in 5 new breast cancer cases will be DCIS. DCIS is often treated by surgery to excise the cancerous tissue, and radiation therapy. In addition, chemotherapy (e.g., tamoxifen) may be used to treat DCIS.

Lobular carcinoma in situ is a pre-cancerous neoplasia. It may be indicative of a predisposition for invasive cancer. LCIS only accounts for about 15% of the in situ (ductal or lobular) breast cancers. Lobular carcinoma in situ is often treated with tamoxifen.

Invasive Ductal Carcinoma (IDC) is the most common invasive breast cancer. As the name implies, it is carcinoma that began in the breast ducts and then invaded the surrounding fatty tissue. About 8 of 10 invasive breast cancers are infiltrating ductal carcinomas. IDC is often treated by surgery to excise the cancerous tissue, and radiation therapy. In addition, chemotherapy (e.g., tamoxifen and trastuzumab) is often used to treat IDC. If the tumor is larger than 4 cm, a radial mastectomy may be performed.

Invasive lobular carcinoma (ILC) is a cancer that develops in the lobules of the breast and has invaded the surrounding tissue. About 1 invasive breast cancer in 10 is an ILC. ILC is treated by surgery to excise the cancerous tissue, and radiation therapy. In addition, chemotherapy (e.g., tamoxifen and trastuzumab) is often used as an adjuvant therapy to treat IDC.

Inflammatory breast cancer accounts for about 1% to 3% of all breast cancers. In inflammatory breast cancer, cancer cells block lymph vessels in the skin resulting in the breast turning read and feeling warm. The affected breast may become larger or firmer, tender, or itchy.

Inflammatory breast cancer is treated with chemotherapy, radiation therapy, and in some cases surgery.

ER+ breast cancer is characterized by the presence of estrogen receptors on the surface of the cancerous cells. Growth of ER+ cancer cells is associated with the availability of estrogen. Treatment options for ER+ breast cancer chemotherapeutic agents that block estrogen (e.g. tamoxifen).

HER2+ breast cancers are characterized by an excess of HER2 on the cell surface of the cancerous cells. HER2+ cancer is often treated with trastuzumab in combination with additional chemotherapeutic agents.

Triple-negative breast cancer is a breast cancer characterized by cells which lack estrogen receptors and progesterone receptors, and do not have an excess of the HER2 protein on their surfaces. Triple-negative breast cancers are often more invasive than other breast cancers. Because the tumor cells lack estrogen and progesterone receptors, hormone therapy (e.g., tamoxifen) is not effective. Additionally, as the cells lack the HER2 protein, drugs that target HER2 (e.g., trastuzumab) are ineffective.

### Methods

Disclosed herein, in certain embodiments, are methods of delivering a composition to a breast duct of an individual in need thereof, comprising: (a) contacting a composition contained within a treatment chamber of a device with a nipple of a breast; and (b) applying positive pressure on the composition. In some embodiments, the composition is forced into a breast duct due to the positive pressure.

Disclosed herein, in certain embodiments, are methods of treating a breast cancer, comprising: (a) contacting a composition comprising at least one therapeutic agent contained within a treatment chamber of a device with a nipple of a breast; and (b) applying positive pressure on the composition comprising at least one therapeutic agent. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

Disclosed herein, in certain embodiments, are methods of diagnosing a disorder of a breast in an individual in need thereof, comprising: (a) contacting a composition comprising a diagnostic agent contained within a treatment chamber of a device with a nipple of a breast; and (b) applying positive pressure on the composition comprising a diagnostic agent. In some embodiments, the breast cancer is a benign breast lesion. In some embodiments, the benign breast lesion is ductal hyperplasia, lobular hyperplasia, atypical ductal hyperplasia, or atypical lobular hyperplasia. In some embodiments, the breast disorder is a breast cancer. In some embodiments, the breast cancer is ductal carcinoma in situ, lobular carcinoma in situ, invasive (or infiltrating) ductal carcinoma, invasive (or infiltrating) lobular carcinoma, or inflammatory breast cancer. In some embodiments, the breast cancer is ER+ breast cancer, HER2+ breast cancer, or triple-negative breast cancer. In some embodiments, the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

In some embodiments, the composition (therapeutic or diagnostic) is instilled into the treatment chamber by injecting it through the second opening (e.g., via a syringe operatively connected to the opening, for example via a luer system). In some embodiments, the composition comprises a therapeutic agent. In some embodiments, the composition comprises a plurality of therapeutic agents. In some embodiments, the composition comprises a diagnostic agent.

In some embodiments, positive pressure is applied to the composition (therapeutic or diagnostic). In some embodiments, the positive pressure is applied to the composition (therapeutic or diagnostic) by introducing a gas into the treatment chamber (e.g., via a syringe operatively connected to the opening, for example via a luer system). In some embodiments, the positive pressure is applied to the composition (therapeutic or diagnostic) by the escape of carbon dioxide from the composition (therapeutic or diagnostic) as the temperature of the composition (therapeutic or diagnostic) increases.

In some embodiments, where the composition comprises a therapeutic agent, the composition is contacted with the nipple of a breast according a predetermined schedule for the therapeutic agent. As the therapeutic agent is being administered topically, the dosage and administration schedule may differ from that used for systemic administration. It is within the knowledge of the skilled artisan to determine an appropriate dosage schedule for the therapeutic agent. In some embodiments, the composition is contacted with the nipple of a breast on the 2^{nd} week of a female individual's menstrual cycle.

In some embodiments, the composition (therapeutic or diagnostic) is contacted with the nipple of a breast for at least 1 hr, 2 hrs, 3 hrs, 4 hrs, 5 hrs, 6 hrs, 8 hrs, 10 hrs, 12 hrs, 18 hours, or 24 hours. In some embodiments, the composition (therapeutic or diagnostic) is contacted with the nipple of a breast overnight.

In some embodiments, the method further comprises anesthetizing the nipple. In some embodiments, the nipple is contacted with a topical anesthetic. In some embodiments, the topical anesthetic comprises lidocaine. In some embodiments, the topical anesthetic is EMLA Cream (lidocaine 2.5% and prilocaine 2.5%), or Topicaine (4% lidocaine or 5% lidocaine).

In some embodiments, the methods further comprise cleaning the nipple before the composition (therapeutic or diagnostic) is contacted with the nipple. The nipple is cleaned by any suitable method. In some embodiments, the nipple is sterilized. In some embodiments, debris (e.g., keratin plugs) is removed from the nipple, increasing access to ducts of the nipple. In some embodiments, the nipple is scrubbed with a mild scrub with a dekeratinizing gel. In some embodiments, the nipple is scrubbed with an exfoliant. Any suitable exfoliant may be used with the methods disclosed herein. Examples of suitable exfoliants include, but are not limited to, microfiber cloths, adhesive exfoliation sheets, micro-bead facial scrubs, crepe paper, crushed apricot kernel or almond shells, sugar or salt crystals, pumice, and abrasive materials such as sponges, loofahs, brushes, salicylic acid, glycolic acid, fruit enzymes, citric acid, malic acid, alpha hydroxy acids (AHAs), and beta hydroxy acids (BHAs). In some embodiments, cleaning the nipple results in the opening of ducts of the nipple. In some embodiments, the ducts of a nipple are about 0.1 to about 0.3 mm in diameter after cleaning.

In some embodiments, the methods further comprise applying a cover over the nipple after removing the device. In some embodiments, the cover is waterproof and/or airtight. In some embodiments, the cover comprises a liquid bandage. In some embodiments, the cover comprises a wound dressing, e.g., a bandage or a patch. In some embodiments, the cover comprises a film. In some embodiments the cover comprises an occlusive agent (e.g., petroleum jelly, mineral oil, shea butter, lanolin, paraffin, beeswax, squalene, triglycerides, coconut oil, sunflower oil, sesame oil, soybean oil, jojoba oil, evening primrose oil and olive oil). In some embodiments, the cover comprises an anti-inflammatory agent or an antiseptic agent.

### Devices

Disclosed herein, in certain embodiments, are methods of delivering a composition to a breast duct of an individual in need thereof, comprising: (a) contacting a composition contained within a treatment chamber of a device with a nipple of a breast; and (b) applying positive pressure on the composition.

The device is constructed of any suitable material. In some embodiments, the device is made of a rigid material. In some embodiments, the device is made of a flexible material. In some embodiments, the device is made of a rigid plastic. In some embodiments, the device is made of a flexible plastic. Any FDA approved material may be used with the devices disclosed herein. In some embodiments, the device is transparent.

In some embodiments, the device comprises a treatment chamber. In some embodiments, the treatment chamber is a hollow receptacle. The treatment chamber is any suitable shape or size which will allow it to operatively cover a nipple of a breast.

The treatment chamber is sized such that it is able to cover a nipple and hold between about 0.5 cc and 10 cc of a composition described herein. In some embodiments, the treatment chamber is sized such that it is able to cover a nipple and hold between about 0.5 cc and 5 cc of a composition described herein. In some embodiments, the treatment chamber is sized such that it is able to cover a nipple and hold between about 0.5 cc and 4 cc of a composition described herein. In some embodiments, the treatment chamber is sized such that it is able to cover a nipple and hold between about 0.5 cc and 3 cc of a composition described herein. In some embodiments, the treatment chamber is sized such that it is able to cover a nipple and hold between about 0.5 cc and 2 cc of a composition described herein. In some embodiments, the treatment chamber is sized such that it is able to cover a nipple and hold about 1 cc and 2 cc of a composition described herein.

In addition to being sized in order to hold a therapeutically-effective or diagnostically-effective volume of the desired composition, in some embodiments, the treatment chamber is sized such that it is able to contain a sufficient volume of headspace (ullage) which may be filled with a sufficient volume of a desired gas, for example, to increase the positive pressure on the composition.

In some embodiments, the device further comprises: a first opening sized to operative cover (or, circumscribe) a nipple, which opening is operatively connected to the treatment chamber. In some embodiments, the first opening is has any shape that is suitable for placement over a nipple. In some embodiments, the first opening is circular in shape. In some embodiments, the first opening allows the treatment chamber to be placed over and in operative contact with a nipple. The inner shape of the first opening does not need to be the same as the outer shape of the opening.

In some embodiments, the first opening is sized such that it circumscribes all or part of an areola or a nipple. In some embodiments, the first opening has a diameter of less than or about 50 mm. In some embodiments, the first opening has a diameter of less than or about 40 mm. In some embodiments, the first opening has a diameter of less than or about 30 mm. In some embodiments, the first opening has a diameter of less than or about 25 mm. In some embodiments, the first opening has a diameter of less than or about 20 mm. In some embodiments, the first opening has a diameter of less than or about 15 mm. In some embodiments, the first opening has a diameter of about 10 mm.

In some embodiments, the device further comprises: a second opening operatively connected to the treatment chamber through which through which the composition is instilled into the treatment chamber. In some embodiments, the second opening is a port. In some embodiments, the opening comprises a seal that inhibits or prevents backflow of the composition out of the treatment chamber. In some embodiments, the second opening is shaped such that a syringe may be operatively connected to the second opening. In some embodiments, the syringe and the second opening connect via a luer system. For example, the syringe may have a male luer lock connection fitting which is able to screw into a female luer lock fitting of the second opening, or alternatively, the syringe may have a female luer lock connection fitting which is able to screw into a male luer lock fitting of the second opening.

In some embodiments, the device further comprises a third opening operatively connected to the treatment chamber through which positive pressure is applied to the composition. In some embodiments, positive pressure is applied by filling the headspace of the treatment chamber with a gas. In some embodiments, the gas is instilled into the treatment chamber via a syringe which operatively connects to the third opening. In some embodiments, the third opening is a port. In some embodiments, the opening comprises a seal that inhibits or prevents loss the gas out of the treatment chamber. In some embodiments, the third opening is shaped such that the syringe is operatively connected to the opening. In some embodiments, the syringe and the third opening connect via a luer system. For example, the syringe may have a male luer lock connection fitting which is able to screw into a female luer lock fitting of the second opening, or alternatively, the syringe may have a female luer lock connection fitting which is able to screw into a male luer lock fitting of the third opening.

In some embodiments, the second opening allows for the installation of the composition and the application of the positive pressure (e.g., the installation of the gas). Where the second opening allows for the installation of the composition and the application of the positive pressure (e.g., the installation of the gas), a third opening may not be required.

In some embodiments, the device further comprises an adhesive which adheres the device to the breast. In some embodiments, the adhesive is any medically suitable skin adhesive. In some embodiments, the skin adhesive is applied to skin before the device is contacted with the skin. In some embodiments, the adhesive is applied to the device after the device has been contacted with the skin. In some embodiments, the adhesive creates a water tight and/or air tight seal.

In some embodiments, the adhesive secures the device to the skin for at least 24 hours. In some embodiments, the adhesive secures the device to the skin for at least 18 hours. In some embodiments, the adhesive secures the device to the skin for at least 12 hours. In some embodiments, the adhesive secures the device to the skin for at least 8 hours. In some embodiments, the adhesive secures the device to the skin for at least 6 hours.

Suitable skin adhesives include, but are not limited to, 2-Octyl (SecureSeal™) skin adhesive, n-Butyl (Liquiband®) skin adhesive, Dow Corning® 9700 Soft Skin Adhesive Parts A & B, Dow Corning® MG 7-9800 Soft Skin Adhesive Parts A & B, Dow Corning® MG 7-9850 Soft Skin Adhesive Parts A & B, Dow Corning® MG 7-9900 Soft Skin Adhesive Parts A & B. In some embodiments, the skin adhesive is a silicone-based skin adhesive. In some embodiments, the skin adhesive is a rubber-based skin adhesive. In some embodiments, the adhesive is a tape or membrane.

### Compositions

Disclosed herein, in certain embodiments, are methods of delivering a composition to a breast duct of an individual in need thereof, comprising: (a) contacting a composition contained within a treatment chamber of a device with a nipple of a breast; and (b) applying positive pressure on the composition. In some embodiments, the composition is forced into the breast duct due to the positive pressure. In some embodiments, the composition comprises at least one therapeutic agent. In some embodiments, the composition comprises a plurality of therapeutic agents. In some embodiments, the composition comprises at least one diagnostic agent. In some embodiments, the composition comprises a plurality of diagnostic agents.

In some embodiments, the composition has a low viscosity at room temperature (between about 20° C. and 25° C.). In some embodiments, the viscosity of the composition at room temperature is suitable for transpapillary penetration.

In some embodiments, the composition has a viscosity of between about 5000 and about 0.5 cp at room temperature. In some embodiments, the composition has a viscosity of between about 2500 and about 0.5 cp at room temperature. In some embodiments, the composition has a viscosity of between about 1000 and about 0.5 cp at room temperature. In some embodiments, the composition has a viscosity of between about 750 and about 0.5 cp at room temperature. In some embodiments, the composition has a viscosity of between about 500 and about 0.5 cp at room temperature. In some embodiments, the composition has a viscosity of between about 250 cp and about 0.5 cp at room temperature. In some embodiments, the composition has a viscosity of between about 100 cp and about 0.5 cp at room temperature. In some embodiments, the composition has a viscosity of between about 50 cp and about 0.5 cp at room temperature. In some embodiments, the composition has a viscosity of between about 10 cp and about 0.5 cp at room temperature. In some embodiments, the composition has a viscosity of between about 5 cp and about 0.5 cp at room temperature. In some embodiments, the composition has a viscosity of between about 1 cp and about 0.5 cp at room temperature.

In some embodiments, the composition has a viscosity of less than 100 cp at room temperature. In some embodiments, the composition has a viscosity of less than 50 cp at room temperature. In some embodiments, the composition has a viscosity of less than 25 cp at room temperature. In some embodiments, the composition has a viscosity of less than 10 cp at room temperature. In some embodiments, the composition has a viscosity of less than 5 cp at room temperature. In some embodiments, the composition has a viscosity of less than 1 cp at room temperature. In some embodiments, the composition has a viscosity of less than 0.5 cp at room temperature.

In some embodiments, the composition is an oil-in-water emulsion in which therapeutics which are poorly soluble in water are dissolved in the oil. In some embodiments, the oil-in-water emulsion comprises an oil that is compatible for treatment of breast conditions. Suitable oils to use with the oil-in-water emulsion include, but are not limited to, soybean oil, medium-chain triglycerides, olive oil, and fish oils. Ins some embodiments, the oil-in-water emulsion is selected from Intralipid®, Liposyn® III, Ivelip®, Lipovenoes®, Lipovenoes® 10% PLR, Intralipos® 10%, Lipofundin-N®, Soyacal, Intrafat, Structolipid® 20%, Lipofundin® MCT/LCT, Lipovenoes® MCT, ClinOleic® 20%, Lipoplus®, SMOFlipid®, and Omegaven®.

### Therapeutic Agents

Without being bound by a particular theory of operation, precancerous hyperplasia of the breast is “driven” by a number of processes. A significant process is the contribution of stimulation of the estrogen/progesterone hormonal axis. Each menstrual cycle, during the proliferative phase and especially week two of the cycle, blood levels of estrogen increase significantly, driving ductal cell division and growth. Following ovulation, if fertilization does not occur, there is involution of the ductal and lobular changes and return to quiescence until the next cycle. Estrogen from systemic sources, mostly the ovaries, as well as local synthesis within the breast from the action of aromatase on testosterone contribute to the growth. A second major stimulation is the generalized effect of a pro-inflammatory environment. This has been considered by some to be the effect of stromal effects on the ductal epithelium. A third stimulation involves the role of “metabolic” drivers, such as glucose driven metabolism and high mitochondrial activity in the process. Finally, HER2 stimulation and oncogene and tumor promoter activation can contribute to either inducing hyperplasia or sustaining it.

Given the drivers of precancerous hyperplasia, certain classes of effectors may be used to reverse the hyperplasia. For example, estrogen receptor antagonists, like tamoxifen or raloxifene, may block the effects of the estrogen surge. In the case of tamoxifen, it is known in the art that metabolites of tamoxifen, especially 4-hydroxytamoxifen which is 100 times more potent than tamoxifen, are likely to be the active moiety (with tamoxifen acting as a prodrug). Since the metabolism of tamoxifen to active derivatives is conducted by the liver, the direct administration methods of the instant patent suggest using tamoxifen metabolites, particularly 4-hydroxy-tamoxifen, in the compositions. In a similar vein, while aromatase inhibitors such as are exemestane are contraindicated in premenopausal women because they raise estrogen by their action in the hypothalamus, these local aromatase inhibitors in conjunction with a tamoxifen analogue like 4-hydroxy-tamoxifen could have synergistic effects.

Preventing breast cancer is possible with selective estrogen receptor (ER) modulators and aromatase inhibitors, which reduce the risk of invasive disease by up to 65% (up to 73% for ER-positive and no effect for ER-negative cancer) and the risk of preinvasive disease [ductal carcinoma in situ (DCIS)] by up to 50%. A growing body of work (including recent preclinical and clinical data) support targeting the HER family [epidermal growth factor receptor (EGFR), or human epidermal growth factor receptor (HER) 1 or ErbB 1) and HER2, HER3, and HER4] for preventing ER-negative and possibly ER-positive breast cancer. Preclinical studies of HER family-targeting drugs in mammary neoplasia show suppression of (i) ER-negative tumors in HER2-overexpressing mouse strains, (ii) ER⁻ tumors in mutant Brca1/p53 [symbol] /_mice, and (iii) ER-positive tumors in the methylnitrosourea (MNU) rat model; tumors arising in both the MNU and mutant Brca1/p53 [symbol] /_models lack HER2 overexpression. Clinical trials include a recent placebo-controlled phase IIb presurgical trial of the dual EGFR HER2 inhibitor lapatinib that suppressed growth of breast premalignancy [including atypical ductal hyperplasia (ADH) and DCIS] and invasive cancer in patients with early-stage, HER2-overexpressing or -amplified breast cancer. These results suggest that effect previously observed in a mouse model of HER2-overexpressing, ER-negative mammary cancer.

The inflammatory target in hyperplasia is thought to be the COX-2 enzyme and therefore COX-2 inhibitors should be useful.

In some embodiments, the therapeutic agent is an anthracycline (e.g., doxorubicin or epirubicin), a platinum agent, a taxane (e.g., paclitaxel or docetaxel), or combinations thereof. In some embodiments, the therapeutic agent is ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, or combinations thereof.

In some embodiments, the therapeutic agent is tamoxifen or a tamoxifen derivative (such as 4-hydroxytamoxifen, N-desmethyltamoxifen and cis-tamoxifen). In some embodiments, the therapeutic agent is butyric acid. In some embodiments, the therapeutic agent is doxorubicin. In some embodiments, the therapeutic agent is epirubicin. In some embodiments, the therapeutic agent is paclitaxel. In some embodiments, the therapeutic agent is docetaxel.

In some embodiments, the therapeutic agent is a combination therapy. Where combination therapy is administered, each of the agents may be administered in combination with any other agent (e.g., simultaneously) or alone. Further, all of the agents may be administered according to the claimed method. Alternatively, some of the agents may be administered according to the claimed method, while others are administered systemically.

In some embodiments, the combination therapy is CAF: cyclophosphamide, doxorubicin, and 5-FU. In some embodiments, the combination therapy is TAC: docetaxel, doxorubicin, and cyclophosphamide In some embodiments, the combination therapy is AC→T: doxorubicin and cyclophosphamide followed by paclitaxel or docetaxel. In some embodiments, the combination therapy is FEC:→T: 5-FU, epirubicin, and cyclophosphamide followed by docetaxel or paclitaxel. In some embodiments, the combination therapy is TC: docetaxel and cyclophosphamide In some embodiments, the combination therapy is TCH: docetaxel, carboplatin, and trastuzumab for HER2/neu positive tumors. In some embodiments, the combination therapy is CMF: cyclophosphamide, methotrexate, and 5-fluorouracil. In some embodiments, the combination therapy is A→CMF: doxorubicin, followed by CMF. In some embodiments, the combination therapy is EC: epirubicin and cyclophosphamide In some embodiments, the combination therapy is AC: doxorubicin and cyclophosphamide

### Diagnostic Agents

### Fluorescent Agents

In some embodiments, the diagnostic agent is a fluorescent agent. All fluorescent agents are encompassed within the term “fluorescent agent.” Specific examples of fluorescent agents given herein are illustrative and are not meant to limit the fluorescent agents for use with the methods disclosed herein.

In some embodiments, the fluorescent agent is a fluorescent dye. In some embodiments, the fluorescent dye is a xanthene (e.g., rhodamines, rhodols and fluoresceins, and their derivatives); bimane; coumarin and their derivatives (e.g., umbelliferone and aminomethyl coumarins); aromatic amine (e.g., dansyl; squarate dyes); benzofuran; fluorescent cyanine; indocarbocyanine; carbazole; dicyanomethylene pyrane; polymethine; oxabenzanthrane; xanthene; pyrylium; carbostyl; perylene; acridone; quinacridone; rubrene; anthracene; coronene; phenanthrecene; pyrene; butadiene; stilbene; porphyrin; pthalocyanine; lanthanide metal chelate complexes; rare-earth metal chelate complexes; and derivatives of such dyes.

In some embodiments, the fluorescent agent is a fluorescein dye. In some embodiments, the fluorescein is selected from the group consisting of: 5-carboxyfluorescein, fluorescein-5-isothiocyanate, fluorescein-6-isothiocyanate and 6-carboxyfluorescein.

In some embodiments, the fluorescent agent is a rhodamine dye. In some embodiments, the rhodamine dye is selected from the group consisting of: tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride.

In some embodiments, the fluorescent agent is a cyanine dye. In some embodiments, the cyanine dye is selected from the group consisting of: Cy3, Cy3B, Cy3.5, Cy5, Cy5.5, Cy7, IRDYE680, Alexa Fluor 750, IRDye800CW, and ICG.

The fluorescent agent is detected by any suitable method. In some embodiments, the fluorescent agent is excited with the appropriate wavelength of light and the resulting fluorescence is detected by microscopy, visual inspection, photographic film, use of electronic detectors such as charge coupled devices (CCDs), photomultipliers, etc.

### Radiocontrast Agents

In some embodiments, the diagnostic agent is a radiocontrast agent. As used herein, “radiocontrast agent” means any contrast agent which enables visualization of internal breast structures, e.g., breast ducts, via X-ray based imaging techniques such as computed tomography (CT) and radiography.

In some embodiments, the radiocontrast agent is an iodine compound. In some embodiments, the iodine compound is ionic. In some embodiments, the iodine compound is nonionic. In some embodiments, the contrast agent is acetrizoic acid, adipiodone (iodipamide), calcium iopodate, diatrizoate, diatrizoic acid (amidotrizoic acid; 3,5-diacetamido-2,4,6-triiodobenzoic acid; Hypaque; Gastrografin; Urografin), diodone, iobenzamic acid, iobitridol (Xenetix 300), iocarmic acid, iocetamic acid, iodixanol (Visipaque), iofendylate, ioglicic acid, ioglycamic acid, iohexol (Omnipaque), iomeprol, iopamidol (Iopamiro, Isovue, Iopamiron, and Niopam), iopanoic acid, iopentol, iopodate sodium (Oragrafin or Gastrografin), iopromide (Ultravist), iopydol, iotalamic acid, iotrolan (Isovist), iotroxic acid, ioversol, ioxaglic acid (Hexabrix), ioxilan (Oxilan), ioxitalamic acid (Telebrix), lipiodol (ethiodized oil; Ethiodol), methiodal, metrizamide, metrizoic acid, propyliodone (Dionosil), sodium iodamide, tyropanoic acid (Bilopaque, Lumopaque, Tyropaque, Bilopac), or any combinations thereof.

### MRI Contrast Agents

In some embodiments, the diagnostic agent is a MRI contrast agent. As used herein, “MRI contrast agent” means any contrast agent which enables visualization of internal breast structures, e.g., breast ducts, via magnetic resonance imaging (MRI).

In some embodiments, the MRI contrast agent is a gadolinium (III) containing agent. In some embodiments, the MRI contrast agent is gadobenate (MultiHance), gadobutrol (Gadovist), gadodiamide (Omniscan), gadofosveset (Ablavar, formerly Vasovist), gadopentetate (Magnevist, Magnegita, Gado-MRT ratiopharm), gadoterate (Dotarem), gadoteridol (ProHance), gadoversetamide (OptiMARK), gadoxetate (Primovist, Eovist), or any combinations thereof.

In some embodiments, the MRI contrast agent is a gadolinium chelate. In some embodiments, the MRI contrast agent is diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), or combinations thereof.

In some embodiments, the MRI contrast agent is an iron oxide containing agent. In some embodiments, the MRI contrast agent is superparamagnetic iron oxide or ultrasmall superparamagnetic iron oxide. In some embodiments, the MRI contrast agent is ferucarbotran (Resovist), feruglose (Clariscan), ferumoxides injectable solution (Feridex I.V.), ferumoxsil (Lumirem), ferumoxtran (Combidex, Sinerem), or any combinations thereof.

In some embodiments, the MRI contrast agent is superparamagnetic iron platinum.

In some embodiments, the MRI contrast agent is paramagnetic manganese.

### Ultrasound Contrast Agents

In some embodiments, the diagnostic agent is an ultrasound contrast agent. As used herein, “ultrasound contrast agent” means any contrast agent which enables visualization of internal breast structures, e.g., breast ducts, via ultrasound. In some embodiments, the ultrasound contrast agent is a microbubble. In some embodiments, the ultrasound contrast agent perflexane lipid microspheres (Imagent, Imavist), perflutren lipid microspheres (Definity), galactose microparticles (Levovist), perflutren protein-type A microspheres (Optison), or any combinations thereof. In some embodiments, the ultrasound contrast agent is conjugated to a targeting moiety.

### Radionuclides

In some embodiments, the diagnostic agent is a nuclear probe. In some embodiments, the diagnostic agent is a SPECT or PET radionuclide probe. In some embodiments, the radionuclide probe is selected from: a technetium chelate, a copper chelate, a radioactive fluorine, a radioactive iodine, and an indium chelate.

In some embodiments, the diagnostic agent is HYNIC, DTPA, and DOTA. In some embodiments, the diagnostic agent is ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu, a radioactive isotope of Lu, or any combinations thereof.

### Additional Components

In some embodiments, the composition comprises a dissolved gas. In some embodiments, the gas a high solubility in a cold liquid (e.g., between about 0° C. and 5° C.) and a low solubility in a liquid at room temperature. In some embodiments, the gas is carbon dioxide, oxygen, nitrogen, or any combinations thereof. In some embodiments, the gas is carbon dioxide. In some embodiments, the gas is oxygen. In some embodiments, the gas is nitrogen.

In some embodiments, the composition is refrigerated so that the dissolved gas stays in solution. In some embodiments, the composition is stored between 0° C. and 20° C. In some embodiments, the composition is stored between 0° C. and 15° C. In some embodiments, the composition is stored between 0° C. and 10° C. In some embodiments, the composition is stored between 0° C. and 5° C. In some embodiments, the composition is stored between 0° C. and 4° C. In some embodiments, the composition is stored between 0° C. and 2° C. In some embodiments, the composition is stored between 0° C. and 1.6° C.

What is claimed is:

1. A method of delivering a composition to a breast duct of an individual in need thereof, comprising:
a. contacting a composition contained within a treatment chamber of a device with a nipple of a breast; and
b. applying positive pressure on the composition.

2. The method of claim 1, wherein the composition is forced into the breast duct due to the positive pressure.

3. The method of claim 1, wherein the device further comprises: a first opening sized to circumscribe a nipple, which opening is operatively connected to the treatment chamber.

4. The method of claim 1, wherein the device further comprises: a second opening operatively connected to the treatment chamber through which through which the composition is instilled into the treatment chamber.

5. The method of claim 1, wherein the device further comprises a third opening operatively connected to the treatment chamber through which positive pressure is applied to the composition.

6. The method of claim 1, wherein the composition comprises at least one therapeutic agent.

7. The method of claim 1, wherein the composition comprises a plurality of therapeutic agents.

8. The method of claim 1, wherein the composition comprises at least one therapeutic agent selected from the group consisting of: an anthracycline, a platinum agent, a taxane, or combinations thereof.

9. The method of claim 1, wherein the composition comprises at least one therapeutic agent selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, and combinations thereof.

10. The method of claim 1, wherein the composition comprises 4-hydroxytamoxifen.

11. The method of claim 1, wherein the composition comprises tamoxifen.

12. The method of claim 1, wherein the composition comprises N-desmethyltamoxifen.

13. The method of claim 1, wherein the composition comprises cis-tamoxifen.

14. The method of claim 1, wherein the composition comprises butyric acid.

15. The method of claim 1, wherein the composition comprises doxorubicin.

16. The method of claim 1, wherein the composition comprises epirubicin.

17. The method of claim 1, wherein the composition comprises paclitaxel.

18. The method of claim 1, wherein the composition comprises docetaxel.

19. The method of claim 1, wherein the composition comprises fluorouracil.

20. The method of claim 1, wherein the composition comprises at least one diagnostic agent.

21. The method of claim 1, wherein the composition comprises a plurality of diagnostic agents.

22. The method of claim 1, wherein the composition comprises a diagnostic agent selected from a fluorescent agent, a contrast agent and a radionuclide.

23. The method of claim 1, wherein the composition comprises a fluorescent agent selected from the group consisting of: a fluorescein dye, a rhodamine dye and a cyanine dye.

24. The method of claim 1, wherein composition comprises a diagnostic agent selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE680, Alexa Fluor 750, IRDye800CW, ICG.

25. The method of claim 1, wherein the composition comprises a contrast agent selected from a radiocontrast agent, MRI contrast agent, or ultrasound contrast agent.

26. The method of claim 22, wherein the composition comprises a contrast agent selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof.

27. The method of claim 22, wherein composition comprises a radionuclide selected from the group consisting of: ²¹¹At, ¹³¹I, ¹²⁵I, ⁹⁰Y, ¹⁸⁶Re, ¹⁸⁸Re, ¹⁵³Sm, ²¹²Bi, ³²P, ^{64 Cu}, a radioactive isotope of Lu, or any combinations thereof.

28. The method of claim 20, further comprising detecting the diagnostic agent.

29. The method of claim 1, wherein the composition has a low viscosity.

30. The method of claim 1, wherein the composition has a viscosity of less than 10 cp, 5 cp, or 1 cp at 25° C.

31. The method of claim 1, wherein the composition comprises dissolved carbon dioxide.

32. The method of claim 1, wherein the composition is stored between 0° C. and 20° C.

33. The method of claim 1, wherein the positive pressure is applied to the composition by the escape of the carbon dioxide from the composition as the temperature of the composition increases.

34. The method of claim 1, wherein the composition is contacted with the nipple of a breast on the 2^{nd} week of the individual's menstrual cycle.

35. The method of claim 1, wherein the composition is contacted with the nipple of a breast for at least 6 hrs, 8 hrs, 10 hrs, 12 hrs, 18 hours, or 24 hours.

36. The method of claim 1, further comprising adhering the device to the nipple.

37. The method of claim 1, wherein the device further comprises an adhesive which adheres the device to the breast.

38. The method of claim 1, further comprising applying a topical anesthetic to the nipple before the composition is contacted with the nipple.

39. The method of claim 1, further comprising cleaning the nipple before the composition is contacted with the nipple.

40. The method of claim 1, further comprising applying a cover over the nipple after removing the device.

41. The method of claim 40, wherein the cover is waterproof and/or airtight.

42. The method of claim 40, wherein the cover comprises a liquid bandage.

43. The method of claim 40, wherein the cover comprises a patch.

44. The method of claim 40, wherein the cover comprises a film.

45. The method of claim 40, wherein the cover comprises an occlusive agent.

46. The method of claim 40, wherein the cover comprises an anti-inflammatory agent or an antiseptic.

47. A method of treating a breast disorder, comprising:
a. contacting a treatment chamber comprising a composition comprising at least one therapeutic agent with a nipple of a breast; and
b. applying positive pressure on the composition comprising at least one therapeutic agent.

48. The method of claim 47, wherein the breast disorder is a breast cancer.

49. The method of claim 48, wherein the breast cancer is ductal carcinoma in situ, lobular carcinoma in situ, invasive (or infiltrating) ductal carcinoma, invasive (or infiltrating) lobular carcinoma, or inflammatory breast cancer.

50. The method of claim 48, wherein the breast cancer is triple-negative breast cancer.

51. The method of claim 48, wherein the breast cancer is adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma, or micropapillary carcinoma.

52. The method of claim 47, wherein the composition comprising at least one therapeutic agent is forced into the breast duct due to the positive pressure.

53. The method of claim 47, wherein the device further comprises:
a. a first opening sized to circumscribe a nipple, which opening is operatively connected to the treatment chamber; and
b. a second opening operatively connected to the treatment chamber through which positive pressure is applied to the composition comprising at least one therapeutic agent.

54. The method of claim 47, wherein the device further comprises a third opening through which the composition comprising at least one therapeutic agent is instilled into the treatment chamber.

55. The method of claim 47, wherein the composition comprises a plurality of therapeutic agents.

56. The method of claim 47, wherein the at least one therapeutic agent is selected from the group consisting of: an anthracycline, a platinum agent, a taxane, or combinations thereof.

57. The method of claim 47, wherein the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen, toremifene, trastuzumab, vinorelbine, and combinations thereof.

58. The method of claim 47, wherein the at least one therapeutic agent is hydroxytamoxifen.

59. The method of claim 47, wherein the composition comprises tamoxifen.

60. The method of claim 47, wherein the composition comprises N-desmethyltamoxifen.

61. The method of claim 47, wherein the composition comprises cis-tamoxifen.

62. The method of claim 47, wherein the at least one therapeutic agent is butyric acid.

63. The method of claim 47, wherein the at least one therapeutic agent is doxorubicin.

64. The method of claim 47, wherein the at least one therapeutic agent is epirubicin.

65. The method of claim 47, wherein the at least one therapeutic agent is paclitaxel.

66. The method of claim 47, wherein the at least one therapeutic agent is docetaxel.

67. The method of claim 47, wherein the at least one therapeutic agent is fluorouracil.

68. The method of claim 47, further comprising sealing the device to the nipple.

69. The method of claim 47, further comprising cleaning the nipple before the treatment chamber is contacted with the nipple.

70. The method of claim 47, further comprising applying a cover over the nipple after removing the device.

71. A method of diagnosing a disorder of a breast in an individual in need thereof, comprising:
a. contacting a treatment chamber comprising a composition comprising a diagnostic agent with a nipple of a breast; and
b. applying positive pressure on the composition comprising a diagnostic agent.

72. The method of claim 71, whereby the composition comprising a diagnostic agent is forced into the breast duct due to the positive pressure.

73. The method of claim 71, wherein the device further comprises:
a. a first opening sized to circumscribe a nipple, which opening is operatively connected to the treatment chamber; and
b. a second opening operatively connected to the treatment chamber through which positive pressure is applied to the composition comprising a diagnostic agent.

74. The method of claim 71, wherein the device further comprises a third opening through which the composition comprising a diagnostic agent is instilled into the treatment chamber.

75. The method of claim 71, wherein the diagnostic agent is selected from a fluorescent agent, a contrast agent and a radionuclide.

76. The method of claim 75, wherein the fluorescent agent is selected from the group consisting of: a fluorescein dye, a rhodamine dye and a cyanine dye.

77. The method of claim 71, wherein diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE710, Alexa Fluor 750, IRDye800CW, ICG.

78. The method of claim 75, wherein the contrast agent is a radiocontrast agent, MRI contrast agent, or ultrasound contrast agent.

79. The method of claim 75, wherein the contrast agent is selected from the group consisting of:
acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof.

80. The method of claim 75, wherein the radionuclide is selected from the group consisting of: ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu, a radioactive isotope of Lu, or any combinations thereof.

81. The method of claim 71, further comprising detecting the diagnostic agent.

82. The method of claim 71, further comprising sealing the device to the nipple.

83. The method of claim 71, further comprising cleaning the nipple before the treatment chamber is contacted with the nipple.

84. The method of claim 71, further comprising applying a cover over the nipple after removing the device.

85. A composition for use in the treatment or diagnosis of a breast cancer, comprising (a) at least one therapeutic agent or a diagnostic agent, and (b) a dissolved gas.

86. The composition of claim 85, wherein the dissolved gas is carbon dioxide.

87. The composition of claim 85, wherein the composition has a low viscosity.

88. The composition of claim 85, wherein the composition has a viscosity of less than 10 cp, 5 cp, or 1 cp at 25° C.

89. The composition of claim 85, comprising a plurality of therapeutic agents.

90. The composition of claim 85, wherein the at least one therapeutic agent is selected from the group consisting of: an anthracycline, a platinum agent, a taxane, or combinations thereof.

91. The composition of claim 85, wherein the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, and combinations thereof.

92. The composition of claim 85, wherein the at least one therapeutic agent is 4-hydroxytamoxifen.

93. The composition of claim 85, wherein the at least one therapeutic agent is tamoxifen.

94. The composition of claim 85, wherein the at least one therapeutic agent is N-desmethyltamoxifen.

95. The composition of claim 85, wherein the at least one therapeutic agent is cis-tamoxifen.

96. The composition of claim 85, wherein the at least one therapeutic agent is butyric acid.

97. The composition of claim 85, wherein the at least one therapeutic agent is doxorubicin.

98. The composition of claim 85, wherein the at least one therapeutic agent is epirubicin.

99. The composition of claim 85, wherein the at least one therapeutic agent is paclitaxel.

100. The composition of claim 85, wherein the at least one therapeutic agent is docetaxel.

101. The composition of claim 85, wherein the at least one therapeutic agent is fluorouracil.

102. The composition of claim 85, wherein the diagnostic agent is selected from a fluorescent agent, a contrast agent and a radionuclide.

103. The composition of claim 102, wherein the fluorescent agent is selected from the group consisting of: a fluorescein dye, a rhodamine dye and a cyanine dye.

104. The composition of claim 85, wherein diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE850, Alexa Fluor 750, IRDye800CW, ICG.

105. The composition of claim 102, wherein the contrast agent is a radiocontrast agent, MRI contrast agent, or ultrasound contrast agent.

106. The composition of claim 102, wherein the contrast agent is selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof.

107. The composition of claim 102, wherein the radionuclide is selected from the group consisting of: ²¹¹At, ¹³¹I, ¹²⁵I, ⁹⁰Y, ¹⁸⁶Re, ¹⁸⁸Re, ¹⁵³Sm, ²¹²Bi, ³²P, ⁶⁴Cu, a radioactive isotope of Lu, or any combinations thereof.

108. The composition of claim 85, wherein the composition is stored between 0° C. and 20° C.

109. A device for delivering a composition to a breast duct of an individual in need thereof, comprising:
a. a treatment chamber;
b. a first opening sized to circumscribe a nipple, which opening is operatively connected to the treatment chamber; and
c. a composition comprising at least one therapeutic agent or a diagnostic agent.

110. The device of claim 109, wherein the composition comprising the at least one therapeutic agent or the diagnostic agent is contained within the treatment chamber.

111. The device of claim 109, further comprising a second opening operatively connected to the treatment chamber through which through which the composition is instilled into the treatment chamber.

112. The device of claim 109, further comprising a third opening operatively connected to the treatment chamber through which positive pressure is applied to the composition.

113. The device of claim 109, wherein the composition comprises a plurality of therapeutic agents.

114. The device of claim 109, wherein the at least one therapeutic agent is selected from the group consisting of: an anthracycline, a platinum agent, a taxane, or combinations thereof.

115. The device of claim 109, wherein the at least one therapeutic agent is selected from the group consisting of: ado-trastuzumab emtansine, albumin-bound paclitaxel, anastrozole, capecitabine, carboplatin, cisplatin, cyclophosphamide, docetaxel, doxorubicin HCl, epirubicin HCl, eribulin, everolimus, exemestane, fluorouracil, fulvestrant, gemcitabine HCl, goserelin acetate, ixabepilon, lapatinib ditosylate, letrozole, liposomal doxorubicin, megestrol acetate, methotrexate, mitoxantrone, paclitaxel, pamidronate disodium, pertuzumab, raloxifene, tamoxifen. toremifene, trastuzumab, vinorelbine, and combinations thereof.

116. The device of claim 109, wherein the at least one therapeutic agent is hydroxytamoxifen.

117. The device of claim 109, wherein the at least one therapeutic agent is tamoxifen.

118. The device of claim 109, wherein the at least one therapeutic agent is N-desmethyltamoxifen.

119. The device of claim 109, wherein the at least one therapeutic agent is cis-tamoxifen.

120. The device of claim 109, wherein the at least one therapeutic agent is butyric acid.

121. The device of claim 109, wherein the at least one therapeutic agent is doxorubicin.

122. The device of claim 109, wherein the at least one therapeutic agent is epirubicin.

123. The device of claim 109, wherein the at least one therapeutic agent is paclitaxel.

124. The device of claim 109, wherein the at least one therapeutic agent is docetaxel.

125. The device of claim 109, wherein the at least one therapeutic agent is fluorouracil.

126. The device of claim 109, wherein the composition comprises a plurality of diagnostic agents.

127. The device of claim 109, wherein the diagnostic agent is selected from a fluorescent agent, a contrast agent and a radionuclide.

128. The device of claim 127, wherein the fluorescent agent is selected from the group consisting of: a fluorescein dye, a rhodamine dye and a cyanine dye.

129. The device of claim 109, wherein diagnostic agent is selected from the group consisting of: 5-carboxyfluorescein, fluorescein-5-isothiocyanate, fluorescein-6-isothiocyanate, 6-carboxyfluorescein, tetramethylrhodamine-6-isothiocyanate, 5-carboxytetramethylrhodamine, 5-carboxy rhodol derivatives, tetramethyl and tetraethyl rhodamine, diphenyldimethyl and diphenyldiethyl rhodamine, dinaphthyl rhodamine, rhodamine 101 sulfonyl chloride, Cy3, Cy3B, Cy3.5, Cy5, Cy5.5, Cy7, IRDYE680, Alexa Fluor 750, IRDye800CW, ICG.

130. The device of claim 127, wherein the contrast agent is a radiocontrast agent, MRI contrast agent, or ultrasound contrast agent.

131. The device of claim 127, wherein the contrast agent is selected from the group consisting of: acetrizoic acid, adipiodone (iodipamide), calcium iopodate, diatrizoate, diatrizoic acid (amidotrizoic acid; 3,5-diacetamido-2,4,6-triiodobenzoic acid), diodone, iobenzamic acid, iobitridol, iocarmic acid, iocetamic acid, iodixanol, iofendylate, ioglicic acid, ioglycamic acid, iohexol, iomeprol, iopamidol, iopanoic acid, iopentol, iopodate sodium, iopromide, iopydol, iotalamic acid, iotrolan, iotroxic acid, ioversol, ioxaglic acid, ioxilan, ioxitalamic acid, lipiodol (ethiodized oil), methiodal, metrizamide, metrizoic acid, propyliodone, sodium iodamide, tyropanoic acid, gadobenate, gadobutrol, gadodiamide, gadofosveset, gadopentetate, gadoterate, gadoteridol, gadoversetamide, gadoxetate, diethylene triamine pentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), ferucarbotran, feruglose, ferumoxides injectable solution, ferumoxsil, ferumoxtran, perflexane lipid microspheres, perflutren lipid microspheres, galactose microparticles, perflutren protein-type A microspheres, or any combinations thereof.

132. The device of claim 127, wherein the radionuclide is selected from the group consisting of: ²¹¹At, ¹³¹I, ¹²⁵I, ⁹⁰Y, ¹⁸⁶Re, ¹⁸⁸Re, ⁵¹³Sm, ²¹²Bi, ³²I, ⁶⁴Cu, a radioactive isotope of Lu, or any combinations thereof.

133. The device of claim 109, wherein the composition has a low viscosity.

134. The device of claim 109, wherein the composition has a viscosity of less than 10 cp, 5 cp, or 1 cp at 25° C.

135. The device of claim 109, wherein the composition comprises dissolved carbon dioxide.

136. The device of claim 109, further comprising an adhesive which adheres the device to the breast.
